(19) United States
(12) Patent Application Publication
(10) Pub. No.: US 2011/0059168 A1
(43) Pub. Date: Mar. 10, 2011

(54) METHOD FOR CORRECTING INTESTINAL GLUTAMINE SYNTHETASE DEFICIENCY

(76) Inventors: Sam ANG, Rowland Heights, CA (US); Daniel Kuang Hsien Wu, Taipei (TW)

(21) Appl. No.: 12/556,534

(22) Filed: Sep. 9, 2009

(51) Int. Cl. A61K 9/48; A61K 35/74; A61K 9/64; A61K 9/28

(52) U.S. Cl. 424/456; 424/93.4; 424/463; 424/474

(57) ABSTRACT

The method includes steps of providing non-pathogenic glutamine-synthetase-producing bacteria, introducing the provided non-pathogenic glutamine-synthetase-producing bacteria into intestines, releasing the introduced non-pathogenic glutamine-synthetase-producing bacteria in the intestines in a predetermined time period, and normalizing serum level of free glutamate and halting continual flooding of free glutamate into the brain.

### BACKGROUND OF THE INVENTION

The present invention relates to a method for correcting intestinal glutamine synthetase deficiency.

A need for a method to correct intestinal glutamine synthetase deficiency has been presented. This invention is directed at solving certain neurological and psychotic disorders caused by glutamate toxicity.

### SUMMARY OF THE INVENTION

The present invention contrives to solve the disadvantages of the prior art.

An objective of the invention is to provide a method for correcting intestinal glutamine synthetase deficiency.

Another object of the invention is to provide a method for correcting intestinal glutamine synthetase deficiency, which uses beneficial bacteria such as, but not limited to butyrivibro fibrisolvens and lactobacillus plantarum.

The present invention relates to a method for correcting intestinal glutamine synthetase deficiency using enteric coated non-pathogenic, glutamine synthetase-producing bacteria such as, but not limited to butyrivibro fibrisolvens and lactobacillus plantarum to replace the glutamine synthetase-producing bacteria commonly found in human intestines, including but not limited to gram-negative bacteria such as E. coli, bacteriodes fragilis, pseudomonas and klebsiella spp., and gram-positive bacteria, such as, but not limited to lactobacillus plantarum.

We believe that glutamine synthetase produced by these bacteria in the intestines, is a vital enzyme for converting most of the glutamate from food sources into glutamine, and the deficiency of these bacteria causes serum-free glutamate level to climb beyond 20 times the serum basal level, resulting in a large amount of free glutamate crossing the blood brain barrier. Neuron cells then become calcified and eventually die, and there will also be many other forms of damage such as dysfunctional glutamate transporters and over-reacting glutamate receptors among others, hence a wide spectrum of neurological and psychotic illnesses.

Since most of these glutamine synthetase bacteria found in human intestines are potentially opportunistic pathogens, it is not a good idea to replenish them for product liability issues. We can replace their vital role in the intestines by other beneficial bacteria such as, but not limited to butyrivibro fibrisolvens and lactobacillus plantarum which are also known to produce glutamine synthetase. They can be enteric coated in a capsule, tablet or soft gel form, which protects them from the corrosive effect of gastric acid. Taken orally they will be released in the intestines approximately 1 to 2 hours later, over time they establish themselves in the intestines, producing sufficient glutamine synthetase to convert food source glutamate into glutamine in the intestines, hence limiting the serum level of glutamine to approximately 4.4 to 8.8 mg/L. It will therefore halt the continual or sporadic flooding of free glutamate into the brain after each high-glutamate meal, and over time could halt or even reverse the progress of many types of neurological or mental disorders.

We can of course add glutamine synthetase for better and faster results, as it takes times to establish beneficial bacteria such as, but not limited to butyrivibro fibrisolvens and/or lactobacillus plantarum in the intestines, but currently all glutamine synthetase are regent grade and need to be keep at approximately −20° C., if supplement grade, room-temperature-stable glutamine synthetase can one day be made available, it is better to add enteric coated glutamine synthetase into our solution so that before non-pathogenic glutamine-synthetase-producing bacteria such as butyrivibro fibrisolvens and/or lactobacillus plantarum establish themselves in the intestines, the supplemental glutamine synthetase can immediately help converting food source glutamate into glutamine, and effectively prevent further brain damages by glutamate toxicity.

We believe the root cause of some neurological and psychotic disorders is the deficiency in glutamine synthetase-producing bacteria commonly found in human intestines. These groups of bacteria play a vital role in converting food source glutamate into L-glutamine, and therefore normalize the serum level of glutamate to 4.4 to 8.8 mg/L and prevent flooding of excessive level (higher than 20 folds basal level) of glutamate into the brain and cause damages to various parts of the brain. Any means of restoring room-temperature-stable glutamine synthetase into the intestines, and any means of restoring other forms of pathogenic bacteria (including but not limited to bacteriodes fragilis, bacteriodes fragilis, klebsiella and puedomonas), and non-pathogenic bacteria (including but not limited to butyrivibro fibrisolvens and lactobacillus plantarum) that are known to produce glutamine synthetase though any means into the intestines are in violation of this patent.

An aspect of the invention provides a method for correcting intestinal glutamine synthetase deficiency.

The method comprises steps of: providing non-pathogenic glutamine-synthetase-producing bacteria; introducing the provided non-pathogenic glutamine-synthetase-producing bacteria into intestines; releasing the introduced non-pathogenic glutamine-synthetase-producing bacteria in the intestines in a predetermined time period; and normalizing serum level of free glutamate to a value from about 4.4 to about 8.8 mg/L and halting continual flooding of free glutamate into a brain.

The non-pathogenic glutamine-synthetase-producing bacteria produce glutamine synthetase.

The non-pathogenic glutamine-synthetase-producing bacteria may comprise, but not limited to butyrivibro fibrisolvens, lactobacillus plantarum, or butyrivibro fibrisolvens and lactobacillus plantarum.

The steps of providing glutamine synthetase to the intestines may comprise a step of containing the non-pathogenic glutamine-synthetase-producing bacteria in a capsule, tablet or soft gel. The capsule, the tablet or soft gel may be configured to protect the non-pathogenic glutamine-synthetase-producing bacteria from the corrosive effect of gastric acid.

The predetermined time period may be from about 1.0 to 2.0 hours.

The steps of introducing may comprise a step of orally taking the non-pathogenic glutamine-synthetase-producing bacteria. The steps of providing may comprise a step of containing the non-pathogenic glutamine-synthetase-producing bacteria in a capsule, tablet or a soft gel.

The steps of providing may comprise a step of adding a predetermined amount of glutamine synthetase to the non-pathogenic glutamine-synthetase-producing bacteria.

The predetermined amount of glutamine synthetase may be calculated from an average turnover of food-source glutamate in a typical meal.

The steps of providing may comprise a step of adding a predetermined amount of glutamine synthetase to the intestines orally.

The predetermined amount of glutamine synthetase may be calculated from an average turnover of food-source glutamate in a typical meal.

The added glutamine synthetase may comprise enteric coated, room-temperature-stable glutamine synthetase. The room-temperature-stable glutamine synthetase may be stable for at least one day.

The non-pathogenic glutamine-synthetase-producing bacteria may produce glutamine synthetase.

The steps of introducing may comprise a step of introducing the non-pathogenic glutamine-synthetase-producing bacteria into the patient's intestines if the plasma-free glutamate level of such patient has increased to over 88 mcg/g within two hours of consuming a regular high-protein meal.

The predetermined healthy stable level of plasma-free glutamate may be between about 4.4 mcg/g to 8.8 mcg/g (ppm).

The advantages of the present invention are: (1) the method provides a method for correcting intestinal glutamine synthetase deficiency; and (2) the method uses beneficial bacteria to produce glutamine synthetase in the intestines.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will be better understood with reference to the accompanying drawings, wherein:

FIG. 1 is a flowchart showing a method for correcting intestinal glutamine synthetase deficiency according to an embodiment of the present invention.

### DETAILED DESCRIPTION EMBODIMENTS OF THE INVENTION

An abundant amino acid, glutamate is found in virtually all foods, including meat, fish, poultry, breast milk, vegetables, soy sauce, fish sauce, MSG and oyster sauce. Protein-rich foods such as breast milk, cheese and meat contain large amounts of bound glutamate. Vegetables tend to contain proportionally higher levels of free glutamate, especially peas, tomatoes, and potatoes. A number of early studies with dogs (Neame and Wiseman 1958), and later, studies conducted in rats (Windmueller 1982, Windmueller & Spaeth 1974, 1975) demonstrated that the vast majority (95%) of dietary glutamate is converted into glutamine and then metabolized in the gastrointestinal tract. In fact, very little dietary glutamate enters either the systemic or the portal blood supply (Young and Ajami et al 2000), indicating it is almost exclusively utilized by the intestinal tissues.

Glutamine, one of the most abundant amino acids present in nature, comprises 10-40% of most proteins within our bodies by weight. Glutamine is formed from glutamate (a precursor of glutamine) by the action of glutamine synthetase. As a consequence of the rapid metabolism of glutamate in intestinal mucosal cells, with any excess glutamate being metabolized by the liver, systemic plasma levels remain typically low, even after ingestion of large amounts of dietary protein (Munro 1979, Meister 1979). Human plasma is reported to contain between 4.4-8.8 mg/L (ppm) of free glutamate (Pulce et al 1992).

Newborn babies are able to metabolize free glutamate (22% of total amino acids) from formula milk and 1 year old infants are able to metabolize protein base glutamate as efficiently as adults (filer et al 1979). However, data collected by Lewis, stegink, Filer et al in 1979, in contrast to those by Bizzi et al (1977), indicate a considerable variation (from 2 μmoles/dL to 11 μmoles/dL plasma glutamate) in the absorption and metabolism of both free and peptide bound glutamate by normal adults on a high-protein diet consisting of a serving of egg-milk custard or a hamburger and a milk shake at 1 g/kg.

Glutamine synthesis from glutamate and ammonia by glutamine synthetase represents one of the several quantitatively significant enzymatic mechanisms for the utilization of ammonia (Meister, Harvey lecture 63, 139, 1969).

Human intestines contain several bacteria known to produce glutamine synthetase, including but not limited to gram-negative bacteria such as E. coli, bacteriodes fragilis, pseudomonas and klebsiella spp., and gram-positive bacteria such as, but not limited to lactobacillus plantarum. Among them, E coli appears to be the most common and significant commensal (or an opportunistic pathogen if allowed to overgrow or mutated) bacteria found in most human intestines, yet, over the last 13-year period, we have observed a handful of patients, whose stool cultures were consistently devoid of E coli, due to the fact that most gram-negative bacteria are pathogens, and that broad-spectrum and gram-negative antibiotics had been administered extensively to prevent or treat infection. It is possible that some of these glutamine-synthetase-producing bacteria were weakened to a point where it could not recover without clinical intervention.

While it may not be clinically feasible to accurately determine if a patient lacks glutamine-synthetase-producing bacteria from a stool culture test, and therefore has lost or has a weakened ability to metabolize food-source glutamate, we have discovered that in a statistically significant percentage of psychotic and neurological disorder patients, free glutamate in their urine ranges from 50 mcg/g to as high as 4122.4 mcg/g (ppm), while the normal range should be 15-35 mcg/g (ppm).

In guinea pigs, rats and mice, brain glutamate levels remain unchanged after an administration of large oral doses of MSG which results in plasma levels increasing up to 18-fold (Peng et al 1973, Liebschultz et al 1977, Caccia et al 1982, Airoldi et al 1979, Bizzi et al 1977). Brain glutamate increases significantly only when plasma levels are about 20 times the basal value (about 88 mg/L) following an oral dose of 2 g MSG/kg of body weight (Bizzi et al 1977). Our patients did not ingest or inject MSG, they simply had a high-protein (free of MSG) breakfast, yet their plasma level of free glutamate could potentially or temporarily be as high as 11 to 937 times the basal value. It is obvious that, among some of our psychotic and neurological disorder patients, a temporary or permanent deficiency in glutamine-synthetase-producing bacteria is responsible for this extremely elevated plasma level of free glutamate, high enough to cross blood brain barrier and cause extensive interference or damage to their brain function.

Numerous research papers have linked glutamate toxicity to neurological and psychotic disorders, which include but not limited to following diseases: Alzheimer's disease, Amyotrophic Lateral Sclerosis, Ataxia, Autism, Cerebellum Atrophy, Dementia, Epilepsy, Huntington's disease, Multiple Sclerosis, Obsessive Compulsive Disorder, Parkinson's Disease, Schizophrenia, Stiff Man Syndrom, and Stroke.

Many theories have been proposed for glutamate toxicity in the brain; these include but not limited to gene defects, dysfunctional glutamate transporters, lack of ATP, over-activation of glutamate receptors and elevated level of homocysteine. None, however, can scientifically explain why by consuming a regular high-protein breakfast, urine free glutamate level surges as high as 4122.4 mcg/g while the normal value is expected to be no higher than 35 mcg/g. We conclude that a temporary or permanent deficiency in glutamine-synthetase-producing bacteria, together with high-glutamate diet, which results in the inability to metabolize glutamate effectively, is one of the most significant, scientific and logical explanations of glutamate toxicity in the brain.

We all know bipolar patients “flip-flop” between two personalities, but so far no one has actually offered a logical explanation for such “flip-flop” behaviors. Gene defects, heavy metal toxicity, virus infections, metabolic disorders etc have been proposed, but none offers a convincing explanation for the “flip-flop” changes of psychotic states. Suppose a bipolar patient happens to lack glutamine-synthetase-producing bacteria in the intestines, and therefore unable to metabolize glutamate effectively. A high-protein diet will cause a surge of free glutamate in the plasma level, high enough to cross the blood brain barrier and disrupt the normal function of brain glutamate as a neurotransmitter; therefore the “flip-flopping” from normal to psychotic state occurs. And when excess glutamate is finally removed from the brain, such bipolar patient “flip-flops” back from psychotic to normal state. It is our hope that we can gather enough clinical data in the future to validate such hypothesis.

In one interesting incident, we presented a roast duck to a bipolar patient. After the patient consumed the entire roast duck, which contained at least 36,360 ppm of bound glutamate and 690 ppm of free glutamate, the patient was paralyzed for nearly 17 hours. A stool culture later confirmed the patient lacked certain glutamine-synthetase-producing bacteria.

The present invention relates to a method for correcting intestinal glutamine synthetase deficiency using enteric coated non-pathogenic bacteria such as but not limited to butyrivibro fibrisolvens and/or lactobacillus plantarum to replace the loss of or weakened glutamine-synthetase-producing bacteria in one's intestines. A noticeable percentage of patients of neurological or mental disorders appear to be deficient in glutamine-synthetase-producing bacteria.

The glutamine synthetase produced by some bacteria in the intestines, is a vital enzyme for converting most of the glutamate from food sources into glutamine, and the deficiency of these bacteria causes serum-free glutamate level to climb beyond 20 times the serum basal level, resulting in a large amount of free glutamate crossing the blood brain barrier. Neuron cells then become calcified and eventually die, and there will also be many other forms of damage such as dysfunctional glutamate transporters and over-reacting glutamate receptors among others, hence a wide spectrum of neurological and psychotic illnesses.

Since most of these glutamine synthetase bacteria found in human intestines are potentially opportunistic pathogens, it is not a good idea to replenish them for product liability issues. Their vital role can be replaced in the intestines by other beneficial bacteria such as, but not limited to butyrivibro fibrisolvens and lactobacillus plantarum which are also known to produce glutamine synthetase. They can be enteric coated in a capsule, tablet or soft gel form, which protects them from the corrosive effect of gastric acid. Taken orally they will be released in the intestines about 1 to 2 hours later, over time they establish themselves in the intestines, producing sufficient glutamine synthetase to convert food source glutamate into glutamine in the intestines, hence limiting the serum level of glutamine to approximately 4.4 to 8.8 mg/L. It will therefore halt the continual or sporadic flooding of free glutamate into the brain after each meal, and over time could halt or even reverse the progress of many types of neurological or mental disorders.

It is possible to add glutamine synthetase for better and faster results, as it takes times to establish beneficial bacteria such as butyrivibro fibrisolvens and/or lactobacillus plantarum in the intestines. But so far all the glutamine synthetase is regent grade and needs to be kept at −20° C. If supplement grade, shelf-stable glutamine synthetase can one day be made available. It will be better to add glutamine synthetase into our solution so that before non-pathogenic glutamine-synthetase-producing bacteria such as, but not limited to butyrivibro fibrisolvens and/or lactobacillus plantarum establish themselves in the intestines, the supplemental glutamine synthetase can immediately help converting food source glutamate into glutamine, and effectively prevent further brain damages by glutamate toxicity.

This patent is about our discovery of one of the possible causes of some neurological and mental disorders; that they are caused by the deficiency or complete loss of glutamine-synthetase-producing bacteria in the intestines, which play a key role in converting 95% of food source glutamate into glutamine, and therefore normalize the serum level of glutamate to 4.4 to 8.8 mg/L and prevent flooding of an excessive level (higher than 20 times the basal level) of free glutamate into the brain causing damages to various part of the brain.

Any means of restoring supplemental or pharmaceutical grade room-temperature-stable glutamine synthetase into the intestines, and any means of restoring other forms of pathogenic bacteria (including but not limited to E coli, bacteriodes fragilis, klebsiella and puedomonas), and non-pathogenic bacteria (including but not limited to butyrivibro fibrisolvens and lactobacillus plantarum) that are known to produce glutamine synthetase through any means into the intestines are in violation of this patent.

This invention relates to a method for correcting intestinal glutamine synthetase deficiency using enteric coated non-pathogenic, glutamine-synthetase-producing bacteria such as, but not limited to butyrivibro fibrisolvens and lactobacillus plantarum to replace the glutamine-synthetase-producing bacteria commonly found in human intestines, including but not limited to gram-negative bacteria such as E. coli, bacteriodes fragilis, pseudomonas and klebsiella spp., and gram-positive bacteria lactobacillus plantarum.

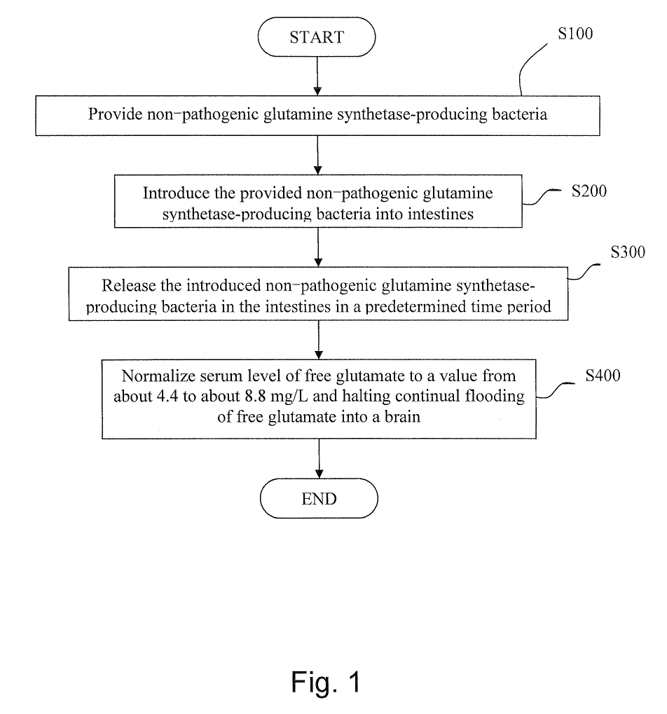

FIG. 1 shows a flowchart illustrating a method for correcting intestinal glutamine synthetase deficiency according to an embodiment of the present invention.

An aspect of the invention provides a method for correcting intestinal glutamine synthetase deficiency.

The method comprises steps of: providing non-pathogenic glutamine synthetase-producing bacteria (S100); introducing the provided non-pathogenic glutamine synthetase-producing bacteria into intestines (S200); releasing the introduced non-pathogenic glutamine synthetase-producing bacteria in the intestines in a predetermined time period (S300); and normalizing serum level of free glutamate to a value from about 4.4 to 8.8 mg/L and halting continual flooding of free glutamate into the brain (S400).

The non-pathogenic glutamine-synthetase-producing bacteria produce glutamine synthetase.

The non-pathogenic glutamine-synthetase-producing bacteria may comprise, but not limited to butyrivibro fibrisolvens, Lactobacillus Plantarum, or butyrivibro fibrisolvens and lactobacillus plantarum. In certain embodiments of the invention, the non-pathogenic glutamine-synthetase-producing bacteria may comprise other types of bacteria other than butyrivibro fibrisolvens and lactobacillus plantarum.

The steps of providing (S100) may comprise a step of containing the non-pathogenic glutamine-synthetase-producing bacteria in a capsule, tablet or a soft gel. The capsule, the tablet or the soft gel may be configured to protect the non-pathogenic glutamine-synthetase-producing bacteria from the corrosive effect of gastric acid.

The predetermined time period may vary from approximately 1.0 to 2.0 hours.

The steps of introducing (S200) may comprise a step of orally taking the non-pathogenic glutamine-synthetase-producing bacteria. The steps of providing (S100) may comprise a step of containing the non-pathogenic glutamine-synthetase-producing bacteria in a capsule, tablet or a soft gel.

The steps of providing (S100) may comprise a step of adding a predetermined amount of glutamine synthetase to the non-pathogenic glutamine-synthetase-producing bacteria.

The predetermined amount of glutamine synthetase may be calculated from an average turnover of glutamate in a typical meal.

The steps of providing (S100) may comprise a step of adding a predetermined amount of glutamine synthetase to the intestines orally.

The predetermined amount of glutamine synthetase may be calculated from an average turnover of glutamate in a typical meal.

The added glutamine synthetase may comprise enteric coated, room-temperature-stable glutamine synthetase. The room-temperature-stable glutamine synthetase may be stable for at least one day.

The non-pathogenic glutamine-synthetase-producing bacteria may produce glutamine synthetase.

The steps of introducing (S200) may comprise a step of introducing the non-pathogenic glutamine-synthetase-producing bacteria into the patient's intestines if the plasma-free glutamate level of such patient has increased to over 88 mcg/g within two hours of consuming a regular high-protein meal.

The predetermined healthy stable level of plasma-free glutamate may be between approximately 4.4 mcg/g to 8.8 mcg/g (ppm).

In certain embodiments of the invention, the predetermined amount of glutamine synthetase may be calculated from the average daily turnover of glutamate in a 70 kg man estimated at 4,800 mg.

While the invention has been shown and described in reference to different embodiments thereof, it will be appreciated by those skilled in the art that variations in form, detail, composition and operation may be made without departing from the spirit and scope of the invention as defined by the accompanying claims.

What is claimed is:

1. A method for correcting intestinal glutamine synthetase deficiency, the method comprising steps of:
providing non-pathogenic glutamine-synthetase-producing bacteria;
introducing the provided non-pathogenic glutamine-synthetase-producing bacteria into intestines;
releasing the introduced non-pathogenic glutamine-synthetase-producing bacteria in the intestines in a predetermined time period; and
normalizing serum level of free glutamate to a value approximately between 4.4 and 8.8 mg/L and halting continual flooding of free glutamate into the brain,
wherein the non-pathogenic glutamine-synthetase-producing bacteria produce glutamine synthetase.

2. The method of claim 1, wherein the non-pathogenic glutamine-synthetase-producing bacteria comprise butyrivibro fibrisolvens.

3. The method of claim 1, wherein the non-pathogenic glutamine-synthetase-producing bacteria comprise lactobacillus plantarum.

4. The method of claim 1, wherein the non-pathogenic glutamine-synthetase-producing bacteria comprise butyrivibro fibrisolvens and lactobacillus plantarum.

5. The method of claim 4, wherein the steps of providing comprises a step of containing the non-pathogenic glutamine-synthetase-producing bacteria in a capsule, tablet or a soft gel.

6. The method of claim 5, wherein the capsule, the tablet or the soft gel is configured to protect the non-pathogenic glutamine-synthetase-producing bacteria from the corrosive effect of gastric acid.

7. The method of claim 6, wherein the predetermined time period is approximately between 1.0 and 2.0 hours.

8. The method of claim 1, wherein the steps of introducing comprises a step of orally taking the non-pathogenic glutamine-synthetase-producing bacteria.

9. The method of claim 8, wherein the step of providing comprises a step of containing the non-pathogenic glutamine-synthetase-producing bacteria in a capsule, tablet or a soft gel.

10. The method of claim 1, wherein the step of providing comprises a step of adding a predetermined amount of glutamine synthetase to the non-pathogenic glutamine-synthetase-producing bacteria.

11. The method of claim 10, wherein the predetermined amount of glutamine synthetase is calculated from an average turnover of glutamate in a typical meal.

12. The method of claim 1, wherein the step of providing comprises a step of adding a predetermined amount of glutamine synthetase to the intestines orally.

13. The method of claim 12, wherein the predetermined amount of glutamine synthetase is calculated from an average turnover of glutamate in a typical meal.

14. The method of claim 13, wherein the added glutamine synthetase comprises enteric coated, room-temperature-stable glutamine synthetase.

15. The method of claim 14, wherein the enteric coated room-temperature-stable glutamine synthetase is stable for at least one day.

16. The method of claim 1, wherein the non-pathogenic glutamine-synthetase-producing bacteria produce glutamine synthetase.

17. The method of claim 1, wherein the step of introducing comprises a step of introducing the non-pathogenic glutamine-synthetase-producing bacteria into the patient's intestines if the plasma-free glutamate level of such patient has increased to over 88 mcg/g within two hours of consuming a regular high-protein meal.

18. The method of claim 17, wherein the predetermined healthy stable level of plasma-free glutamate is between approximately 4.4 mcg/g and 8.8 mcg/g (ppm).
